(19) United States
(12) Patent Application Publication
(10) Pub. No.: US 2025/0319302 A1
(43) Pub. Date: Oct. 16, 2025

(54) STIMULATING ASSEMBLY FOR A MEDICAL DEVICE

(71) Applicant: Cochlear Limited, Macquarie University, NSW (AU)

(72) Inventors: Marcus Swift, Epping, NSW (AU); Nicholas Charles Kendall Pawsey, North Ryde, NSW (AU)

(21) Appl. No.: 18/862,989

(22) PCT Filed: May 4, 2023
(86) PCT No.: PCT/IB2023/054667

§ 371 Date: Nov. 5, 2024

Related U.S. Application Data
(60) Provisional application No. 63/340,571, filed on May 11, 2022.

(51) Int. Cl. A61N 1/05; A61N 1/36; A61N 1/372

(52) U.S. Cl. CPC A61N 1/0541; A61N 1/36038; A61N 1/37229; A61N 1/36046

(57) ABSTRACT

Presented herein are medical devices that include a stimulating assembly to deliver electrical stimulation to a recipient. More specifically, a stimulating assembly comprises an electrically insulating carrier member and at least one electrode structure coupled to the carrier member. The electrode structure comprises an electrode contact, an electrically conductive polymer pad, and an electrically insulating polymer at least one electrically insulating polymer skirt at least partially surrounding the electrically conductive polymer pad.

### BACKGROUND

### Field of the Invention

The present invention relates generally to a stimulating assembly for a medical device that can be implanted and/or worn by an individual to facilitate stimulation of neurons within the individual's body.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, an apparatus is provided. The apparatus comprises: a carrier member comprising an electrically insulating material; at least one electrode contact; and at least one electrically conductive polymer pad disposed on the electrode contact; and at least one electrically insulating polymer skirt at least partially surrounding the electrically conductive polymer pad.

In another aspect, a system (e.g., a medical device system or other system) comprises the apparatus (e.g., a stimulating assembly) and an electrical stimulator that provides electrical current to the apparatus.

In a further aspect, a method is provided. The method comprises: inserting a stimulating assembly into a body chamber of a recipient, the stimulating assembly comprising a carrier member and an electrode structure coupled with the carrier member, the carrier member comprising an electrically insulating material, and the electrode structure comprising an electrically conductive polymer pad and an electrically insulating polymer skirt at least partially surrounding the pad; and facilitating current flow from the electrode structure to nerve cells within the body chamber.

In another aspect, an implantable stimulating assembly is provided. The implantable stimulating assembly comprises: an electrically insulting carrier member; and an array of electrode structures coupled to carrier member, wherein each of the electrode structures comprises: an electrode contact disposed in the carrier member and having an exposed surface, a conductive polymer pad disposed on exposed surface of the electrode contact, and a polymer skirt disposed on the surface of the carrier member adjacent to exposed surface of the electrode contact.

The above and still further features and advantages of the present invention will become apparent upon consideration of the following detailed description of specific embodiments thereof.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2A is a view in plan of a portion of an elongate stimulating assembly in the cochlear implant system of FIG. 1A;

FIG. 2B is a view in cross-section of the stimulating assembly and, more particularly an electrode structure, taken along lines 2B-2B in FIG. 2A;

FIGS. 3A, 3B and 3C are schematic cross-sectional views depicting stimulation of neurons via electrode structures, in accordance with certain embodiments presented herein.

FIG. 4A is a schematic cross-sectional view depicting stimulation of neurons via a conventional stimulating assembly;

FIG. 4B is a schematic view in cross-sectional view depicting stimulation of neurons via an electrode structure, in accordance with certain embodiments presented herein;

FIG. 4C is another schematic view in cross-sectional view depicting stimulation of neurons via an electrode structure, in accordance with certain embodiments presented herein;

FIG. 5 is a schematic cross-sectional view depicting stimulation of neurons via of a plurality of electrode structures of a stimulating assembly, in accordance with certain embodiments presented herein;

FIGS. 6A, 6B, 6C and 6D are cross-sectional views illustrating additional embodiments of electrode structures, in accordance with certain embodiments presented herein;

FIG. 7A is a schematic cross-sectional view illustrating another electrode structure, in accordance with certain embodiments presented herein;

FIG. 7B is a schematic cross-sectional view illustrating another electrode structure, in accordance with certain embodiments presented herein;

FIGS. 7C and 7D schematically depict operation of the electrode structure of FIG. 7B to show elution of a therapeutic substance from the electrode structure as well as current flow from the electrode structure during operation;

FIG. 8 depicts a view in plan of another embodiment of an electrode structure in accordance with certain embodiments presented herein;

FIG. 9 is a schematic diagram illustrating an implantable stimulator system with which aspects of the devices and techniques presented herein can be implemented;

FIG. 10 is a schematic diagram illustrating a vestibular stimulator system with which aspects of the devices and techniques presented herein can be implemented; and

FIG. 11 is a schematic diagram illustrating a retinal prosthesis system with which aspects of the devices and techniques presented herein can be implemented.

### DETAILED DESCRIPTION

Presented herein are medical devices and, in particular, medical devices that include a stimulating assembly to deliver electrical stimulation to a recipient. More specifically, a stimulating assembly comprises an electrically insulating carrier member and at least one electrode structure coupled to the carrier member. The electrode structure comprises an electrode contact, an electrically conductive polymer pad, and an electrically insulating polymer skirt at least partially surrounding the electrically conductive polymer pad.

Merely for ease of description, the devices and techniques presented herein are primarily described with reference to a specific medical device system, namely a cochlear implant system. However, it is to be appreciated that the devices and techniques presented herein may also be partially or fully implemented by other types of implantable or non-implantable medical devices. For example, the devices and techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The devices and techniques presented herein may also be implemented by dedicated tinnitus therapy devices and tinnitus therapy device systems. In further embodiments, the devices and techniques presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

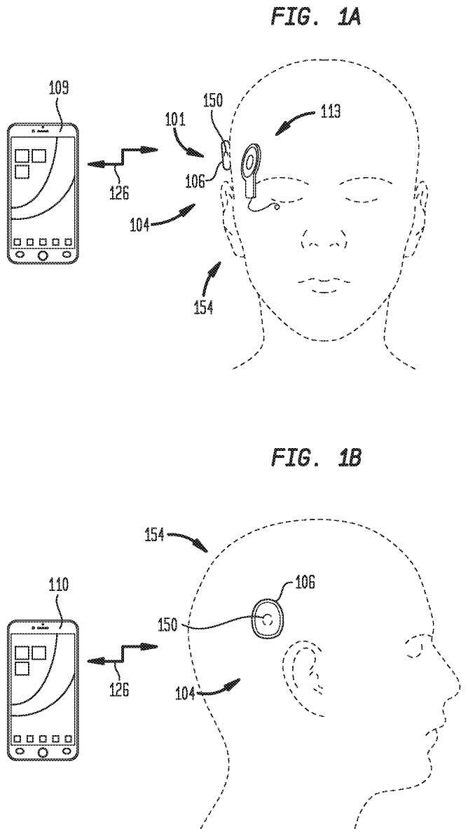

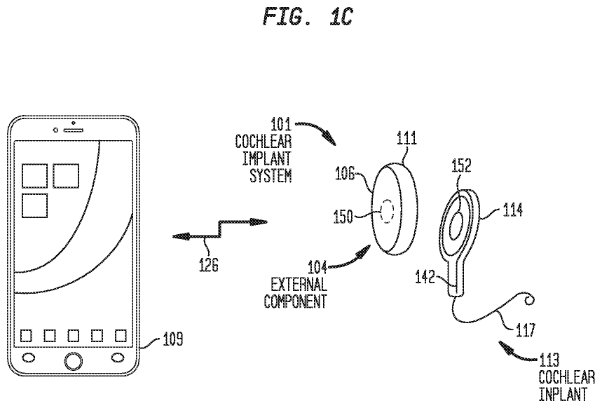

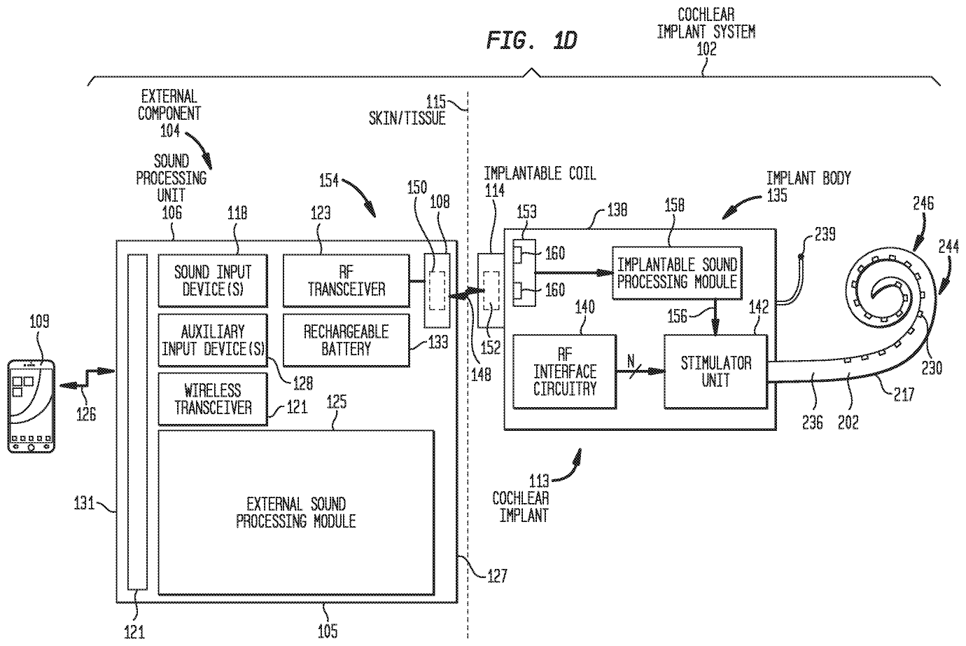

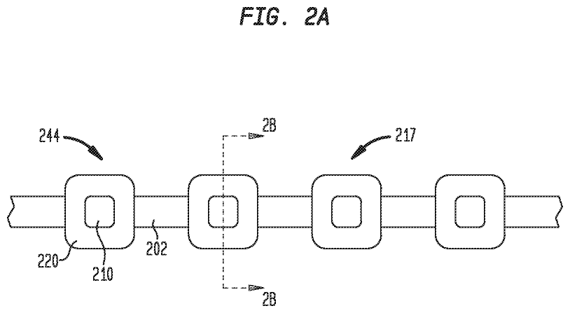

FIGS. 1A-1D illustrates an example cochlear implant system 101 with which aspects of the techniques presented herein can be implemented. The cochlear implant system 101 comprises an external component 104 and an implantable component 113. In the examples of FIGS. 1A-ID, the implantable component is sometimes referred to as a “cochlear implant.” FIG. 1A illustrates the cochlear implant 113 implanted in the head 154 of a recipient, while FIG. 1B is a schematic drawing of the external component 104 worn on the head 154 of the recipient. FIG. 1C is another schematic view of the cochlear implant system 101, while FIG. 1D illustrates further details of the cochlear implant system 101. For ease of description, FIGS. 1A-1D will generally be described together.

Cochlear implant system 101 includes an external component 104 that is configured to be directly or indirectly attached to the body of the recipient and an implantable component 113 configured to be implanted in the recipient. In the examples of FIGS. 1A-ID, the external component 104 comprises a sound processing unit 106, while the cochlear implant 113 includes an implantable coil 114, an implant body 135, and an elongate stimulating assembly 217 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 113. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 113). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 113. For example, in alternative examples, the external component may comprise a behind-the-ear (BTE) sound processing unit or a micro-BTE sound processing unit and a separate external coil assembly. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 101 includes the sound processing unit 106 and the cochlear implant 113. However, as described further below, the cochlear implant 113 can operate independently from the sound processing unit 106, for at least a period, to stimulate the recipient. For example, the cochlear implant 113 can operate in a first general in mode, sometimes referred to as an “external hearing mode,” in which the sound processing unit 106 captures sound signals which are then used as the basis for delivering stimulation signals to the recipient. The cochlear implant 113 can also operate in a second general mode, sometimes referred as an “invisible hearing” mode, in which the sound processing unit 106 is unable to provide sound signals to the cochlear implant 113 (e.g., the sound processing unit 106 is not present, the sound processing unit 106 is powered-off, the sound processing unit 106 is malfunctioning, etc.). As such, in the invisible hearing mode, the cochlear implant 113 captures sound signals itself via implantable sound sensors and then uses those sound signals as the basis for delivering stimulation signals to the recipient. Further details regarding operation of the cochlear implant 113 in the external hearing mode are provided below, followed by details regarding operation of the cochlear implant 113 in the invisible hearing mode. It is to be appreciated that reference to the external hearing mode and the invisible hearing mode is merely illustrative and that the cochlear implant 113 could also operate in alternative modes.

In FIGS. 1A and 1C, the cochlear implant system 101 is shown with an external device 109, configured to implement aspects of the techniques presented. The external device 109 is a computing device, such as a computer (e.g., laptop, desktop, tablet), a mobile phone, remote control unit, etc. As described further below, the external device 109 comprises a telephone enhancement module that, as described further below, is configured to implement aspects of the auditory rehabilitation techniques presented herein for independent telephone usage. The external device 109 and the cochlear implant system 101 (e.g., OTE sound processing unit 106 or the cochlear implant 113) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 121 (e.g., for communication with the external device 109). However, it is to be appreciated that one or more input devices may include additional types of input devices and/or less input devices (e.g., the wireless short range radio transceiver 121 and/or one or more auxiliary input devices 128 could be omitted).

The OTE sound processing unit 106 also comprises the external coil 108, a charging coil 131, a closely-coupled transmitter/receiver (RF transceiver) 122, sometimes referred to as or radio-frequency (RF) transceiver 123, at least one rechargeable battery 133, and an external sound processing module 125. The external sound processing module 125 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 113 comprises an implant body (main module) 135, a lead region 236, and the intra-cochlear stimulating assembly 117, all configured to be implanted under the skin/tissue (tissue) 115 of the recipient. The implant body 135 generally comprises a hermetically-sealed housing 138 in which RF interface circuitry 140 and a stimulator unit 142 are disposed. The implant body 135 also includes the internal/implantable coil 114 that is generally external to the housing 138, but which is connected to the RF interface circuitry 140 via a hermetic feedthrough (not shown in FIG. 1D).

As noted, stimulating assembly 217 is configured to be at least partially implanted in the recipient's cochlea. Stimulating assembly 217 includes a carrier member plurality of longitudinally spaced intra-cochlear electrical stimulating contacts (electrode contacts or electrodes) 230 that collectively form a contact or electrode array 246 for delivery of electrical stimulation (current) to the recipient's cochlea. As described further below, each electrode contact 230 is part of a corresponding electrode structure 244.

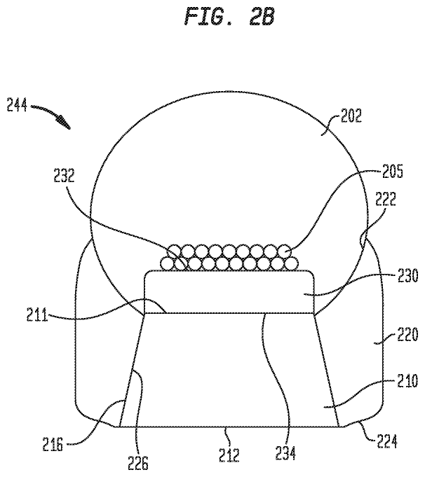

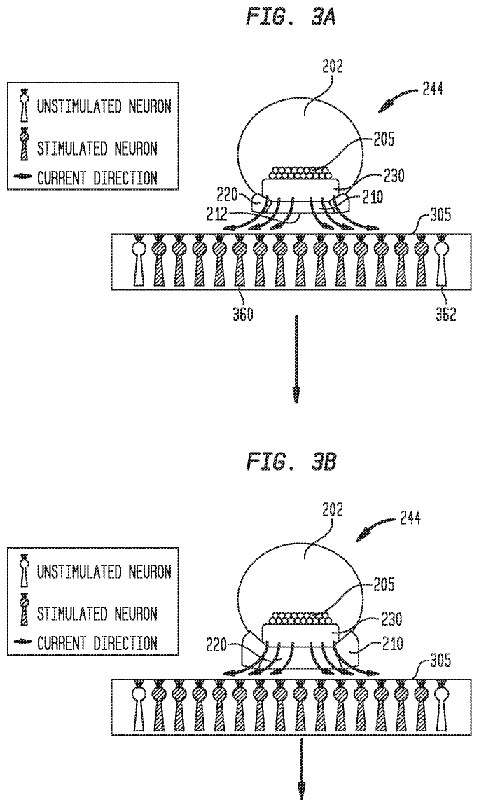

Stimulating assembly 217 extends through an opening in the recipient's cochlea (e.g., cochleostomy, the round window, etc.) and has a proximal end connected to stimulator unit 142 via lead region 236 and a hermetic feedthrough (not shown in FIG. 1D). Lead region 236 includes a plurality of conductors or wires 205 (FIG. 2B) that electrically couple the electrode contacts 230 to the stimulator unit 142. The implantable component 113 also includes an electrode outside of the cochlea, sometimes referred to as the extra-cochlear electrode (ECE) 239.

As noted, the cochlear implant system 101 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 113 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 125. The external sound processing module 125 is configured to convert received input signals (received at one or more of the input devices) into output signals for use in stimulating a first ear of a recipient (i.e., the external sound processing module 125 is configured to perform sound processing on input signals received at the sound processing unit 106). Stated differently, the one or more processors in the external sound processing module 125 are configured to execute sound processing logic in memory to convert the received input signals into output signals that represent electrical stimulation for delivery to the recipient.

As noted, FIG. 1D illustrates an embodiment in which the external sound processing module 125 in the sound processing unit 106 generates the output signals. In an alternative embodiment, the sound processing unit 106 can send less processed information (e.g., audio data) to the implantable component 113 and the sound processing operations (e.g., conversion of sounds to output signals) can be performed by a processor within the implantable component 113.

Returning to the specific example of FIG. 1D, the output signals are provided to the RF transceiver 123, which transcutaneously transfers the output signals (e.g., in an encoded manner) to the implantable component 113 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 101 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 113 receives processed sound signals from the sound processing unit 106. However, in the invisible hearing mode, the cochlear implant 113 is configured to capture and process sound signals for use in electrically stimulating the recipient's auditory nerve cells. In particular, as shown in FIG. ID, the cochlear implant 113 includes a unit 153 including a plurality of implantable sound sensors 160 and an implantable sound processing module 158. Similar to the external sound processing module 125, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 101 could operate differently in different embodiments. For example, in one alternative implementation of the external hearing mode, the cochlear implant 113 could use signals captured by the sound input devices 118 and the implantable sound sensors 160 in generating stimulation signals for delivery to the recipient.

As noted above, the stimulating assembly 217 implanted within the cochlea includes an array or a plurality of electrically stimulating contacts or electrode contacts (electrodes) 230 each forming part of a corresponding electrode structure 244 coupled with (e.g., disposed in/on) an elongated carrier member 202. As shown, e.g., in FIG. 1D, the carrier member 202 has a curved configuration defining a perimodiolar stimulating assembly that is configured to conform with (e.g., wrap around) the modiolus of the cochlea. In particular, the electrode structures 244 are suitably spaced along the carrier member 202 so as to align at suitable positions in relation to the modiolus and neural receptors of the cochlea when the stimulating assembly 217 is implanted within the cochlea.

A portion of the stimulating assembly 117, in which the carrier member 202 is presented in a relatively flat or planar configuration, is depicted in FIG. 2A to show an example embodiment of an array of electrode structures 244 spatially and consecutively aligned along a longitudinal or lengthwise axis of the carrier member 202. The electrode structures 244 can be spatially and consecutively aligned at any selected distances from each other, including equidistant spacing and/or non-equal distance spacing between any two or more pairs of consecutively aligned electrode structures. A cross-sectional view of the assembly 117, taken in a direction transverse the lengthwise axis of the carrier member 202 along line 2B-2B, is depicted in FIG. 2B and shows a cross-sectional view of one of the electrode structures 244 of the assembly. Each of the electrode structures 244 can have the same or similar configuration as the electrode structure depicted in FIG. 2B. Alternatively, any one or more electrode structures can have a different configuration from any one or more other electrode structures in the array provided for the assembly 117.

The carrier member 202 can, in certain embodiments, have a cylindrical configuration with a round or circular cross-section as depicted in FIG. 2B. However, the carrier member can also have any other suitable cross-sectional shape, including polygonal (e.g., square or rectangular) or irregular cross-sectional shapes. The carrier member 202 is further formed of a suitable electrically insulating material, such as a silicone material, that is sufficiently flexible to permit flexion of the assembly 217 from the curved or coiled configuration as depicted in FIG. 1D to an elongated and partially flattened position (e.g., prior to implanting within the recipient's ear), where the carrier member can further be imparted with a structural “memory.” that facilitates curving or coiling of the assembly in the shape as presented in FIG. 1D when at rest (i.e., no flexure force applied to the assembly).

As shown in FIG. 2B, a plurality of electrically conductive wires 205 are completely embedded and extend longitudinally within the carrier member 202 of the assembly 217 and also the lead region 236 so as to electrically couple each electrode structure 244 with the stimulator unit 142 as previously described herein. Thus, the carrier member 202 provides an electrically insulating barrier around the wires 205. Each electrode structure 244 includes an electrode contact 230 that couples with a portion of the wires 205 at the electrode position along the carrier member 202. The electrode contacts 230 and the wires 205 can be formed of the same or different electrically conductive metal (e.g., gold, platinum, a platinum-iridium alloy, etc.) and/or any other suitably electrically conductive material or, alternatively, of different electrically conductive materials.

Each electrode contact 230 is at least partially embedded within the carrier member 202 so as to couple or engage along at least one surface 232 of the contact with the wires 205. As depicted in FIG. 2B, the electrode contact 230 has a generally rectangular configuration and is entirely embedded within the carrier member 202 such that one surface of the contact engages with the wires 205 and an opposing surface is generally aligned with or located slightly below or beneath an exterior peripheral surface portion of the carrier member. As described later, in other embodiments the electrode contact can have other suitable shapes, can be partially embedded within the carrier member but also include a portion that extends beyond the exterior peripheral surface portion of the carrier member. As further depicted in FIG. 2B, a portion of the exterior peripheral surface of the carrier member is removed at the electrode location such that at least a portion of the surface 234 of the electrode contact 230 that opposes the carrier member wire engaging surface 232 is exposed at the corresponding carrier member surface to facilitate transfer of electrical signals between the wires and exterior surface of the carrier member 202.

Each electrode structure 244 further includes an electrically conductive member or conductive pad 210 that is coupled with, but external to, the carrier member 202 and is further positioned over and electrically coupled with the electrode contact 230 at the exposed electrode contact location along the carrier member. The conductive pad 210 can include a contact engaging end 211 that electrically couples and/or engages with the electrode contact surface 234. An insulating peripheral barrier, cup member or skirt 220 is also coupled with the carrier member and is positioned around the conductive pad 210 so as to completely surround the peripheral side portions of the pad. The skirt 220 is open at a terminal or free end 224 so as to enable exposure of a portion, e.g., at least a terminal or free end 212 of the conductive pad 210 at the skirt free end.

Each of the skirt 220 and the pad 210 is formed of a suitably soft or compressive polymer material that is more compressive in relation to the carrier member 202. For example, the skirt and pad can have a hardness or durometer value (e.g., measured using a Shore A durometer scale) that is the same as or less than a hardness of the carrier member. The skirt can be formed of the same insulating silicone material (and thus have the same durometer value) as the carrier member. Alternatively, the skirt can be formed of an insulating silicone material that differs and is softer (lower durometer value) from that of the carrier member. The skirt can also have a hardness or durometer value that is the same or less than that of the pad. For example, the skirt can be formed of a silicone material having a low hardness that provides very soft and compressive or “squishy” properties for the skirt. As described in greater detail herein, the softness of the skirt and the conductive pad are selected so as to facilitate easy deformation and adherence of surface areas of the skirt 220 against the conductive pad 210 as well as surface areas of the pad 210 against the electrode contact 230 in order to provide enhanced sealing effects at the coupling locations/points of contact after implantation of the assembly 217 in the recipient's ear.

The skirt 220 has electrically insulating properties that limit and focus current/electrical signal transfer from the electrode structure 244 to targeted areas as described herein, while the pad 210 is electrically conductive to facilitate transfer of electrical signals from the wires 205 and contacts 230 to neurons within the recipient's body.

An example of an electrically conductive and soft polymer material that is suitable for forming the pad 210 is a mixture of ionomers comprising poly (3,4-ethylenedioxythiophene) (PEDOT). Some non-limiting examples of a soft polymer material that is electrically conductive and can be used to form the conductive pad include a mixture of PEDOT with polystyrene sulfonate (PEDOT:PSS), and a mixture of PEDOT with p-toluenesulfonate (PEDOT:PTS).

One or both of the soft polymers that form the skirt and the pad can further comprise a hydrogel that includes hydrophilic compounds which attract and absorb aqueous fluids (such as perilymph within the cochlea), where the soft polymers swell and expand slightly in volume in response to exposure to and absorption of aqueous fluids. For example, the conductive pad formed of a PEDOT material (e.g., PEDOT:PSS or PEDOT:PTS) can also be implemented as a hydrogel. In some embodiments, only the conductive pad comprises a hydrogel polymer. In other embodiments, both the conductive pad and the skirt can comprise a hydrogel polymer. For example, the skirt can comprise a silicone hydrogel with electrical insulating properties.

The soft, cushioning nature of the insulating skirt and the conductive pad in combination with the shapes or geometries of these two members provides for enhanced surface contact between the skirt and pad as well as between the pad 210 and the electrode contact 230. In the embodiment of FIG. 2B, the skirt 220 has a cup-like or annular shape that at least partially surrounds the pad 210. Surface portions 222 of the skirt 220 also wrap around a portion of the carrier member 202. The surface defining the free end 212 of the conductive pad 210 can extend so as to be generally flush or coplanar with surface portions defining the free end 224 of the skirt 220. In other words, the free end 212 of pad 210 can be configured so as to not be recessed within the skirt 220 but instead flush or generally coplanar with terminal end surface portions of the skirt such that a smooth and continuous transition is defined between the free end 212 of the pad 210 and the skirt free end 224.

The interior, annular surface portions 226 of the skirt 220 engage with complementary outer surface portions 216 of the pad 210 such that the pad fits in a snug or tight engagement within the interior space defined by the skirt. Each of the surface portions 216, 226 define a respective cross-sectional dimension of the pad and the interior space of the skirt. The cross-sectional dimensions of the conductive pad 210 and the interior space of the skirt 220 can also vary so that the outer surface portions 216 of the pad form a wedge within the space between the annular surface portions 226 of the skirt that prevents or significantly limits movement of the pad in relation to the skirt and thus anchors the pad within the skirt and against the electrode contact 230. For example, as shown in FIG. 2B, the outer surface portions 216 of the pad 210 increase or expand in cross-sectional dimension to define a frustoconical shape for the pad that increases in cross-sectional dimension (e.g., diameter) in a direction from its electrical contact engaging end 211 to its free end 212. The space between the interior, annular surface portions 226 of the skirt 220 similarly increases in cross-sectional dimension (e.g., diameter) in a direction that extends from a location corresponding with the electrical contact engaging end 211 of the pad 210 to the skirt free end 224.

The complementary engaging surfaces of the skirt and the pad provide an effective seal for both fluid and electrical current due to the insulating properties of the skirt. In addition, since both the skirt and pad are very soft, these components can compress and conform very effectively to surfaces in which the terminal ends of the skirt and pad are engaged. For example, both the skirt and the pad can be compressed and engage so as to provide sufficient contact with the modiolus wall within the cochlea, including surfaces that may not be very smooth but instead bumpy or having a certain degree of surface roughness, where the soft and/or squishy nature of the skirt and pad maintains contact over flat and uneven surfaces. This minimizes or eliminates the potential for air bubbles to develop between the conductive pad and the modiolus wall as well as maximizing electrical surface area contact between these components. The wedge-like, anchoring engagement between the insulating skirt 220 and the conductive pad 210 and the wrapping of surface portions 222 of the skirt around complementary surface portions of the round or cylindrically shaped elongated carrier member 202 facilitates a tight engagement and adhesion at the interface between the pad 210 (at its end 211) and the electrode contact 230 (at its surface 234).

The electrode structure configurations described herein, which implement an insulating and soft, squishy polymer skirt that surrounds the soft conductive pad and also engages with a portion of the carrier member, enhances the adhesion between the conductive pad and the electrode contact to minimize or prevent any detachment (e.g., delamination) between these components. This further minimizes or prevents air bubbles of fluid gaps at the interface between contact 230 and conductive pad 210 thus enhancing the electrical current transfer at this interface. Further, the liquid absorbing properties of the hydrogel polymer used to form the conductive pad (and optionally the skirt) further enhance sealing of the pad against the body surface (e.g., modiolus wall) of the recipient as well as focusing a direction of current from each electrode structure to a precise surface area of and corresponding neurons associated with the body surface.

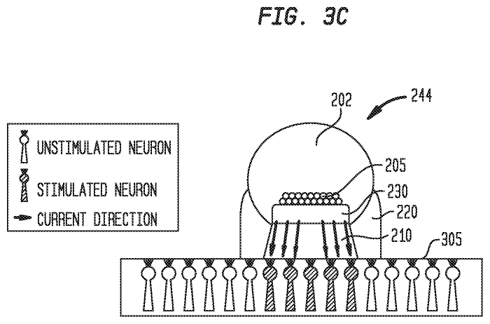

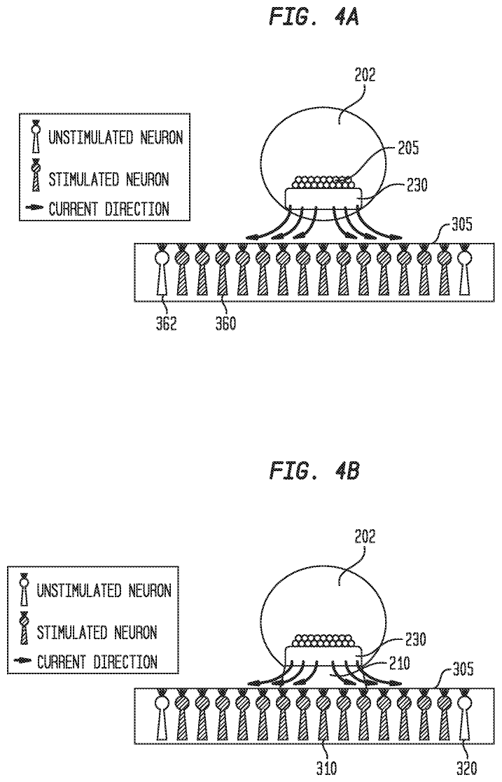

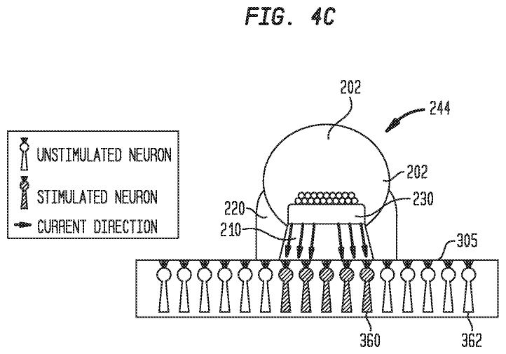

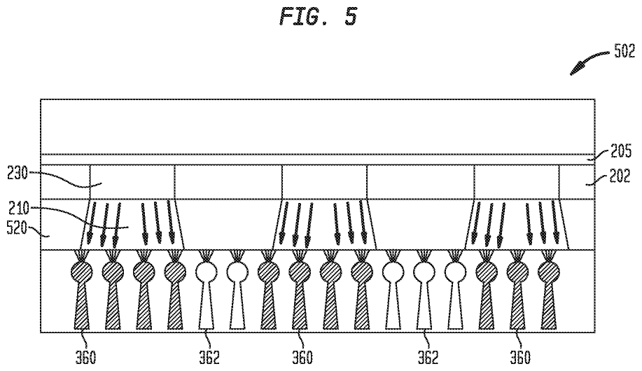

An example embodiment showing operation of electrode structures in a stimulating assembly and where the electrode structures have a configuration of electrode structure 244 is now described with reference to the views schematically depicted in FIGS. 3A-3C. Referring to FIG. 3A, an electrode structure 244 within the array of the stimulating assembly 217 is in a first state after being implanted within the cochlea of the recipient's ear. In this first state, the hydrogel conductive pad 210 is in an original, non-swollen state (i.e., prior to any fluid being absorbed by the hydrogel material) and thus has a smaller profile. In embodiments in which the insulating skirt 220 is also formed of a hydrogel silicone material, the skirt also has a smaller profile prior to implantation of the stimulating assembly. Activation of the cochlear implant transmits electrical impulses or signals from the stimulator unit 142, through the wires 205, and to the electrode contact 230 and conductive pad 210, where current (indicated by the directional arrows) flows from the pad 210 toward the modiolus wall 305 for activating or stimulating neurons 360, 362 at the modiolus wall 305. As schematically depicted in FIGS. 3A-3C (and also FIGS. 4A-4C, 5 and 7A-7D, as described later herein), darker shaded neurons represent stimulated neurons 360 (i.e., neurons activated by the electrical impulses from electrode structure 244) while lighter shaded neurons represent unstimulated neurons 362. Thus, each electrode structure 244, when brought into close proximity with the modiolus wall within the cochlea, stimulates a specified set or number of neurons which are located at an area covered by a flow path of current from the electrode contact 230. Upon initial insertion, while a slight gap might exist between the conductive pad 210, the skirt 220, and the modiolus wall 305, current still flows from the pad 210 to stimulate a set of neurons 360. However, the stimulated neurons 360 include neurons directly beneath the conductive pad 210 as well as adjacent and neighboring neurons that extend beyond an area of the modiolus wall 305 that corresponds with the areal footprint (i.e., the surface area of the free end 212) of the pad.

After a certain period of time within the cochlea, the pad 210 (and optionally the skirt 220) can begin to expand or swell and increase slightly in volume due to absorbing fluid (e.g., perilymph), as is indicated in FIG. 3B. The slight absorption decreases the space or gap between the pad, skirt and the modiolus wall. However, current is still permitted to expand or “leak” beyond the pad and the skirt, thus stimulating a larger area and greater number of neurons 360. Further fluid absorption and swelling occurs until the pad achieves a final volume as depicted in FIG. 3C. In this state, the pad 210 has sufficiently expanded in volume while the pad and skirt 220 also have compressed or deformed slightly at their terminal ends (due to the very soft and/or squishy nature of these components) so as to sufficiently reduce or eliminate the spacing or gap between modiolus wall and the skirt and pad. In particular, the skirt 220 and pad 210 gently deform against the modiolus wall 305 to provide an effective sealing effect at this interface. Due to the insulating properties of the skirt, which surrounds the pad, the flow of current is limited to the surface area of the terminal end of the pad so as activate a smaller number or smaller set of stimulated neurons 360 located in proximity with the electrode structure 244 aligned with the surface area defined at the terminal end 212 of the pad 210.

Thus, the electrode structure configuration described herein directs current flow to a smaller number of neurons in a closely targeted area of the modiolus wall for stimulation and minimizes or prevents current leak to a larger surface area of the modiolus wall due to the sealing nature of the soft and squishy insulating skirt 220 surrounding the pad 210 and engaging the modiolus wall 305. When implemented for use within a person's body (e.g., within the cochlea of the ear), the squishy polymer skirt initially provides very little sealing effect against the body tissue to direct stimulating current from an electrode structure to only a small number of nerve cells or neurons. The current instead spreads out and stimulates all the nerves surrounding the electrode pad for each electrode of the assembly. However, with the absorption of fluid by the hydrogel conductive pad and optionally the skirt), the squishy skirt eventually seals the conductive pad against the body tissue and concentrates the stimulating current to a smaller, more targeted number of neurons. The hydrogel conductive pad also expands over time in the recipient's body to enhance sealing against the body tissue during use. The focusing of electric current by the electrode contact to a smaller number (e.g., more targeted) neurons at the interface with body tissue can also reduce the electrical charge needed to stimulate neurons since current loss that might otherwise occur (due to stimulation of neurons over a wider surface area of body tissue) can be significantly minimized. This can further lower impedance between the electrode contacts and the recipient's body, which can in turn lower electrical energy requirements (e.g., slower drain in battery power) for the cochlear implant or other medical device that implements this electrode structure configuration.

Another feature of implementing the previously described embodiment of the electrode structure 244 in an assembly 217 for a cochlear implant (or other medical device) is a reduction in the trauma or tissue damage associated with implantation of the assembly due to the soft, squishy nature of the skirt for each electrode structure as well as the smaller overall dimensional profile of the hydrogel conductive pad for each electrode during implantation (i.e., the conductive pad has not yet expanded in size). In particular, the pads 210 and skirts 220 of the electrode structures 244 protrude slightly outward as bumps along the elongated carrier member 202 of the assembly 117. The size or profile of these electrode bumps along the carrier member are minimized during the implanting of the cochlear implant device, and the soft or squishy nature of the electrode bumps can compress more easily against tissue surfaces such as the modiolus during such implantation. This combination of features facilitates a more atraumatic surgery for the recipient.

FIGS. 4A-4C also illustrate the effectiveness of the conductive pad/insulating skirt combination of the electrode structure 244 previously described herein in comparison with other types of stimulating assemblies including electrode contacts and/or soft polymer conductive pads. Referring to FIG. 4A, a standard arrangement is depicted including a carrier member 202 and electrode contact 230 that is similar in configuration to that of the electrode structure 244 (as shown in FIG. 4C). However, the arrangement of FIG. 4A does not include a conductive pad or skirt. The arrangement of FIG. 4A has been modified, as shown in FIG. 4B, to include a conductive pad 210 similar to that of the electrode structure 244. However, unlike electrode structure 244, the arrangement of FIG. 4B does not include any insulating skirt that surrounds any portion of the pad 210. Each of the arrangements shown in FIGS. 4A and 4B are operable to stimulate neurons, but the current transmitted from these electrodes is capable of leaking from around the contact 230 and/or the pad 210 so as to increase the surface area of neuron stimulation along the modiolus wall. In particular, the current flow from the electrode contact 230 can still expand beyond the areal footprint of the pad. As shown in FIG. 4C, the skirt 220 of the electrode structure 244 presented herein limits current flow from the electrode to a narrower, more targeted group or number of stimulated neurons 360.

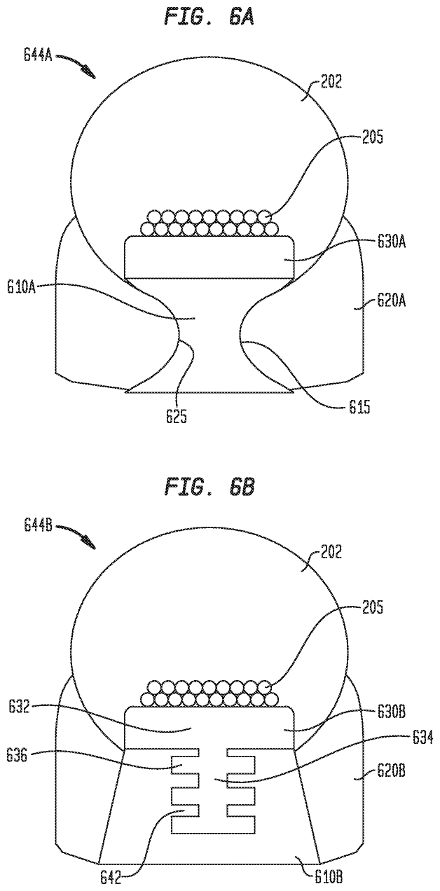

As depicted in FIG. 2A, in certain embodiments, each electrode structure 244 along the carrier member can have the same configuration, where a skirt 220 surrounds a pad 210 for each electrode and where the skirts (aligned with their respective electrodes) are separated from each other along the carrier member. In other embodiments, a single skirt member can be provided in the form of a soft or squishy polymer strip that extends continuously between the electrodes. Referring to FIG. 5, a plurality of electrodes 502 are aligned along a carrier member 202, where each electrode 502 includes an electrode 230 and a conductive pad 210. An insulating skirt member 520 is provided that surrounds the peripheral side portions of each pad 210 and couples with and extends continuously along the carrier member 202 and between the pads. The continuously extending skirt 520 effectively seals against the conductive pads 210 so as to limit current flow to the targeted areas and groups of stimulated neurons 360 that lie in correspondence with an area defined by the interface between the modiolus wall and each pad 210.

The configuration of the electrode structure 244, including geometries/shapes of the insulating skirt, conductive pad and/or electrode contact, can be modified in a number of different ways while still maintaining effective adhesion between pad and electrode contact as well as maintaining and/or maximizing electrical surface contact area between pad and electrode contact and also the pad and body surface of recipient (e.g., modiolus wall for cochlear implant use). In particular, any suitable complementary shapes or geometries at the engaging surfaces/interfaces between the skirt and the conductive pad and/or between the conductive pad and the electrode contact can be implemented that enhance anchoring of the conductive pad with the skirt and also the electrode contact to increase or otherwise enhance the transfer of electrical current between electrode contact and conductive pad while minimizing or preventing air bubbles or air gaps at such engaging interfaces.

For example, the wedge-like engagement between engaging surfaces of the skirt and the pad can be modified so that the pad decreases or tapers in cross-sectional dimension in a direction from its electrical contact engaging end to its free end (with corresponding/complimentary tapering of the interior annular wall surfaces of the skirt). In other words, the tapering or decrease in cross-sectional dimension of the pad can be in a direction opposite of that which is depicted in FIG. 2B.

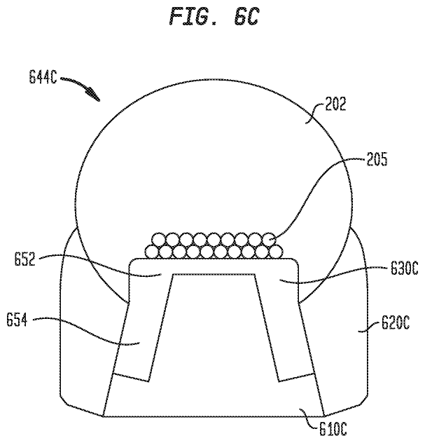

Other example embodiments that show different shapes of the skirt, conductive pad and/or the electrode contact are depicted in FIGS. 6A-6D. Each of the electrode contact, conductive pad and insulating skirt in these embodiments can be formed of the same or similar materials as previously noted for such components of the previously described electrode structure 244.

Referring to FIG. 6A, the electrode structure 644(A) is similar to the previously described electrode structure 244, with the exception that the conductive pad 610(A) and the insulating skirt 620(A) include complementary engaging surfaces that differ from those of the pad 210 and skirt 220 of electrode structure 244. The pad 610(A), which is disposed on electrode contact 630(A), includes a curved and inwardly extending or concave shaped surface portion 615 extending around its lengthwise perimeter, while the skirt 620(A) includes a corresponding or complementary curved and convex shaped surface portion 625 that extends within the annular space defined by the skirt. These geometric configurations of the pad 610(A) and the skirt 620(A) result in larger surface area in which each of the pad and skirt engage with each other. This in turn can enhance sealing between the skirt and the pad, as well as sealing between the pad and electrode contact and between the pad and the body surface (e.g., modiolus wall) of the recipient during use.

In the embodiment of FIG. 6B, the electrode structure 644(B) includes an electrode contact 630(B) that includes a first member in the form of a generally rectangular base member 632 and a second member in the form of an elongated member 634 that extends transversely from the base member 632. The base member 632 is similar in configuration as the electrode contact 230 of the electrode structure 244, where the base member is substantially or entirely disposed within the carrier member 202 and is electrically coupled with the wires 205 extending within the carrier member. The elongated member 634 extends from the base member 632 into a portion of the conductive pad 640, where the conductive pad 610(B) is surrounded by the insulating skirt 220 in a manner similar to that described for electrode structure 244. The elongated member 634 further includes one or more (e.g., a plurality) of cross members 636, where each cross member 634 extends transversely from the elongated member 634 like a spike within the pad 610(B). The pad 610(B) includes a corresponding and complimentary cut-out section suitably dimensioned to receive and seat the elongated member 634 within the pad, where the pad includes corresponding channels that receive the cross members 636 and pillars 642 located between the channels and which extend to the elongated member at locations between the cross members.

The geometric configurations of the electrode contact 630(B) and contact pad 610(B) (which receives and retains the elongated member 634 of the electrode contact), as shown in FIG. 6B, provide an enhanced anchoring interface between electrode contact and conductive pad having a greater surface area than the electrode contact/conductive pad interface for the electrode structure 244. This can enhance adhesion between electrode contact 630(B) and conductive pad 610(B) to minimize or prevent delamination and resultant spaces or gaps between the surfaces at this interface. In addition, the increased surface area between electrode contact and conductive pad can ensure effective transfer of electrical current and reduction of electrical impedance between these components which in turn enhances delivery of electrical signals to neurons for stimulation.

In the embodiment of FIG. 6C, an electrode structure 644(C) is similar to the electrode structure 244 previously described herein with the exception that the electrode contact 630(C) includes a portion that extends between the conductive pad 610(C) and the skirt 620(C). For example, the electrode contact 630(C) includes a base member 652 that electrically couples with the wires 205 and is disposed within the carrier member 202 and one or more sleeve members 654 that extend transversely from the base member 652 around peripheral side portions of the pad 610(C) so as to be located between the conductive pad 610(C) and the skirt 620(C). Thus, the electrode contact 630(C) includes surface area portions along the base member 652 and the sleeve members 654 that electrically couple with the conductive pad 610(C). In a further embodiment, the electrode contact 630(C) can include a sleeve member that extends entirely around a portion of the conductive pad between the pad 610(C) and the skirt 620(C). This configuration increases the contact surface area between electrode contact and conductive pad to provide enhanced attachment and effective transfer of electrical current between these components.

Another example embodiment that can increase surface area contact between the electrode contact and the conductive pad is depicted in FIG. 6D. In this embodiment, the electrode contact 630(C) of electrode structure 644(D) comprises a crisscross or grid/lattice pattern of conductive wiring or a conductive mesh 670 that extends from a base member 653 (attached to wires 205) within the carrier member 202 and is at least partially embedded within the conductive pad 610(D) (where the conductive pad 610(D) is surrounded by the skirt 620(D) in the same manner as described herein form the electrode structure 244). The mesh can extend to any depth within the pad 610(D) to its terminal or free end 682. In the example embodiment depicted in FIG. 6D, the conductive mesh 670 extends within the pad 610(D) to a location in close proximity with, but just beneath, the free end 682 of the pad. This configuration further increases the surface area contact interface between conductive pad and electrode contact to significantly minimize or prevent detachment between the two components at this interface.

Electrode structure configurations in accordance with embodiments presented herein can also be implemented in which engaging surfaces defining an interface between the insulating skirt and the conductive pad and engaging surfaces defining an interface between the conductive pad and the electrode contact can be roughened to any degree of surface roughness that further enhances the adhesion of the engaging surfaces at such interfaces. Roughness can be imparted along the engaging surface by forming bumps or protrusions along these surfaces to increase the surface areas at the interfaces.

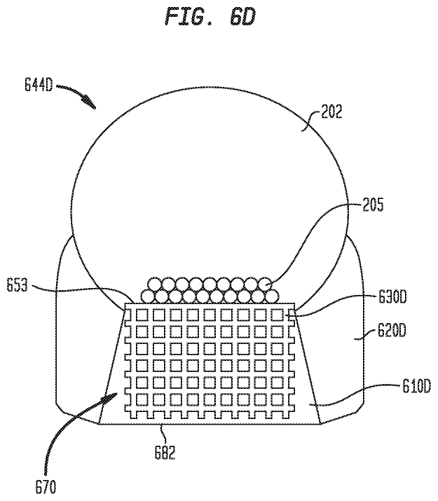

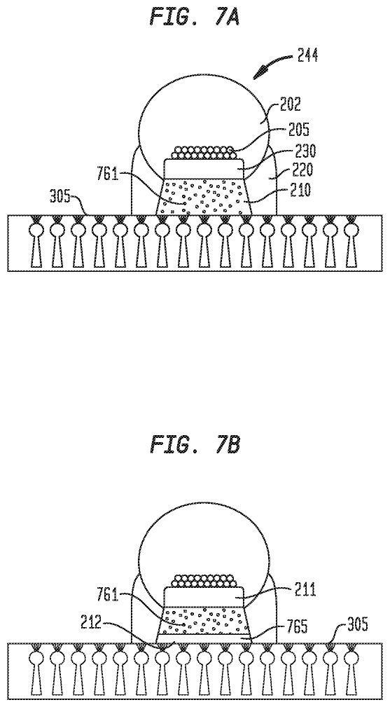

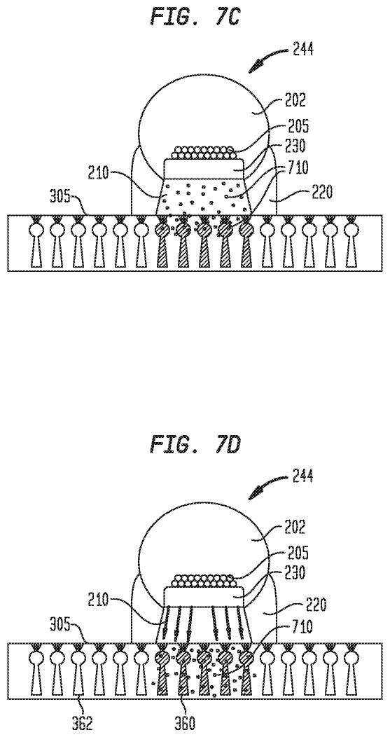

Electrode structures as described herein can be further modified (e.g., loaded/doped) to include a drug or therapeutic substance (also referred to herein as an active ingredient) that is eluted from one or both the conductive pad and the skirt during use and for delivery to body tissues of the recipient. In an example embodiment depicted in FIG. 7A, the therapeutic substance is provided in an electrode structure 244 in the form of solid particles 761 loaded within the hydrogel polymer material of the conductive pad 210. Any suitable therapeutic substance can be provided for localized topical and/or transdermal delivery into a part of the recipient's body. For example, therapeutic substances can be provided to reduce infection (e.g., after a surgical implantation procedure) and/or to promote a specific biological activity within the body of the recipient. In certain embodiments, the conductive pad 210 can be loaded with dexamethasone or another shelf-stable therapeutic substance that can be released following implantation.

The therapeutic substances used herein can include, but are not limited, to large and small molecule therapeutic substances, viral vectors or any other type of known therapeutic substances. That is, in the disclosed embodiments, pharmaceutical compositions may comprise any single or combination of the following therapeutic substances: biological substances. bioactive substances, conjugated or fusion molecules or compounds, viral and non-viral vectors, natural, synthetic and recombinant molecules, antibodies and antibody fragments, etc., pharmaceutical agents/active pharmaceutical ingredients (APIs) including commercially available versions of the same, genes, nucleases, endonucleases, nucleic and ribonucleic acids such as messenger RNA (mRNA), siRNA and miRNA, naked DNA, DNA vectors, oligonucleotides, antisense polynucleotides, peptides, polypeptides, proteins including binding proteins, anti-oxidants, and signalling compounds that promote recovery and resolution, other chemicals, ions, and other molecules used to modulate inflammation within the body of individual. A person of ordinary skill in the art will appreciate that each of these substances can be generated by methods known in the art.

Small molecule therapeutic substances include, without limitation, steroids (e.g., dexamethasone, triamcinolone, fluticasone, prednisolone), antibiotics (including aminoglycoside antibiotics, e.g., Kanamycin, Gentamicin), antiapoptotics, antioxidants, antihistamines, anti-inflammatoires, NSAID (non-steroidal anti-inflammatoires), N-Methyl-D-aspartate (NMDA) receptor antagonists (for treating Tinnitus), therapeutic substance combinations (e.g., FX-322), GSK-3 inhibitors, Wnt activators, sodium thiosulfate (for treating cisplatin-associated ototoxicity, nephrotoxicity and neurotoxicity).

Large molecule therapeutic substances include, without limitation, protein-based therapeutics (therapeutic proteins) including peptides, recombinant proteins, monoclonal antibodies and vaccines, antibody-based therapeutic substances, Fc fusion proteins and other conjugated molecules, anticoagulants, blood factors, bone morphogenetic proteins, engineered protein scaffolds, enzymes, growth factors, hormones including neurotrophins, interferons, interleukins, and thrombolytics.

The therapeutic substance(s) can further be in solid, semi-solid, gel or liquid form, and the particles 761 can be formed substantially from a therapeutic substance or, alternatively, one or more additional substances such as a binder and/or other excipient. Further, release of the particles containing one or more therapeutic substances from the hydrogel conductive pad can be facilitated in any suitable manner. For example, the particles 761 can include a coating over the therapeutic substance that provides a controlled rate of elution and/or delivery of the therapeutic substance from the conductive pad 210 to the modiolus wall 305 (or other tissue surface) of the recipient. The coating can include a water-soluble polymer or other component (e.g., polyethylene glycol, povidone, polyvinyl alcohol, etc.) which, when exposed to an aqueous liquid, has a specified rate of dissolution that releases and allows the therapeutic substance to migrate from the conductive pad 210 toward the modiolus wall 305 as depicted in FIG. 7C. Alternatively, the therapeutic substance itself can be water soluble so as to dissolve in an aqueous liquid and be released from the pad 210. The size of the particles 761 can have suitable dimensions such that the particles are retained within the polymer material of the conductive pad until sufficient dissolution of the particles has been achieved. In addition, when the hydrogel material of the conductive pad absorbs liquid and swells, the porosity of the conductive pad can slightly increase to allow migration of the particles containing therapeutic substance from the pad. Further, the conductive pad can include some sacrificial particles comprising water soluble salts that provide an initial barrier for the drug containing particles within the pad. When the hydrogel material absorbs fluid, the sacrificial particles are dissolved to increase porosity within the pad and facilitate elution of drug containing particles from the pad.

In another embodiment depicted in FIG. 7B, a sacrificial seal/membrane or dissolvable barrier 765 can be provided within or at the terminal or free end 212 of the pad 210 and which extends across (i.e., transverse the lengthwise dimension of) the conductive pad 210. The particles 761 containing the therapeutic substance can be disposed within the pad between the barrier 765 and the contact engaging end 211 of the pad. The dissolvable barrier 765 can be formed of a water-soluble hydrogel or other water-soluble polymer material having a suitable rate of dissolution that, when sufficiently dissolved, facilitates release of the particles 761 from the pad 210 at a specified elution rate. In a further embodiment, the particles including therapeutic substance can be disposed in the barrier 765 such that, when the barrier is sufficiently dissolved, the particles are released from the barrier and pad 210 at a specified elution rate. Further still, the barrier 765 and the conductive pad 210 can each include particles containing the same or different therapeutic substances. For example, the barrier 765 can include a first set of particles including a first therapeutic substance, while the pad 210 can include a second set of particles including a second therapeutic substance. In this example, the first therapeutic substance is eluted for delivery to the recipient prior to elution and delivery of the second therapeutic substance. In still further embodiments, a plurality of barrier layers can be provided (e.g., at the free end of the pad) including one or more different therapeutic substances, having the same or varying thicknesses, made of the same or varying water-soluble polymers and/or having the same or different rates of dissolution (resulting in the same or varying elution rates for the therapeutic substances disposed within each barrier layer).

Referring to FIG. 7C, after a specified time in which the stimulating assembly 217 has been implanted within the cochlea of the recipient and the conductive pad 210 (and optionally the skirt 220) has absorbed fluid (e.g., perilymph) and has swollen, the particles 761 (and/or barrier 765) have sufficiently dissolved by exposure to the fluid causing a release of the particulate material from the pad 210. The particulate material can be absorbed into body tissue at the modiolus wall 305. After some or all of the particulate material including therapeutic substance has eluted from the conductive pad 210 and absorbed by body tissue, electrical current (as shown by the directional arrows in FIG. 7D) can easily flow from the electrode structure 244 to activate neurons 360 in the manner previously described herein.

Thus, the stimulating assembly including electrode structures with configurations as described herein facilitate effective and targeted transfer of electrical current from the electrodes to neurons within body tissue of the recipient while also providing an effective seal between the electrode contact and conductive pad for each electrode during operations.

The electrode structure embodiments have been previously described herein in the context of a stimulating assembly in which electrodes are located in a generally linear and consecutive alignment along a carrier member and the stimulating assembly is implemented in a cochlear implant (e.g., as described in FIGS. 1A-1D and 2A). However, such electrode structures presented herein are also effective for implementation in a variety of different stimulating assembly configurations as well as different types of medical devices for stimulation of nerve cells in body tissue of a recipient, including medical devices in which the stimulating assembly is implanted within a portion of the recipient's body or disposed along an external body surface portion of the recipient.

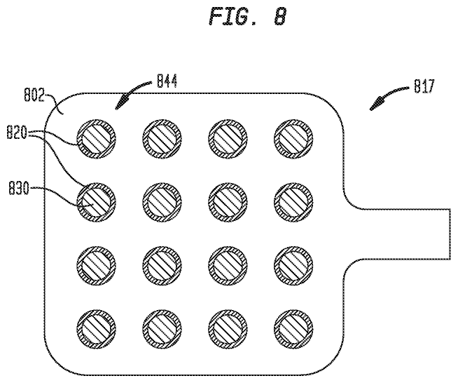

In an example embodiment depicted in FIG. 8, a stimulating assembly 817 is depicted in which a silicone carrier member 802 has a generally flat and rectangular configuration with electrically conductive wiring (not shown) disposed and extending within the carrier member and connecting with a stimulator unit (not shown) that provides electrical signals or impulses to electrode structures 844 disposed in/on the carrier member 802. The electrode structures 844 are aligned along the carrier member 802 in a grid-like manner, with rows and columns of electrodes that are all electrically insulated from each other. The electrode structures 844 can have a similar configuration to electrode structure 244 as depicted in FIG. 2B (i.e., each electrode structures 844 includes an insulating skirt 820 formed of a soft, squishy polymer material that surrounds a hydrogel polymer conductive pad 810 disposed on an electrode contact (not shown in FIG. 8)). While a 4×4 grid of electrode structures 844 is depicted in the example embodiment of FIG. 8, any other pattern or array of electrode structures 844 can also be implemented along a carrier member for a use in a particular medical device.

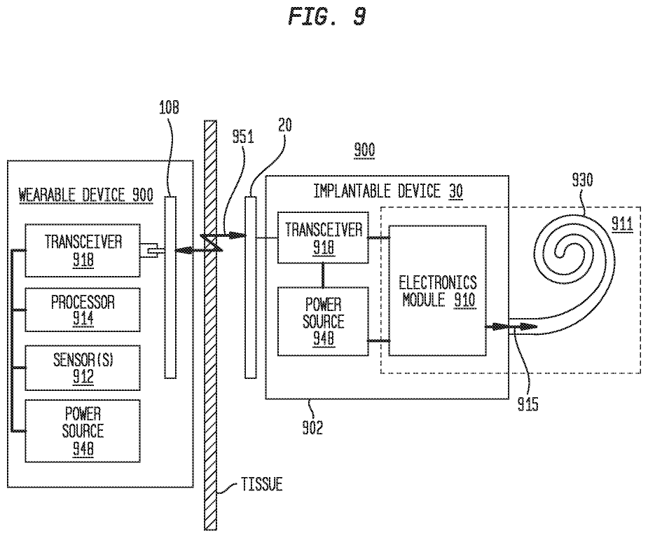

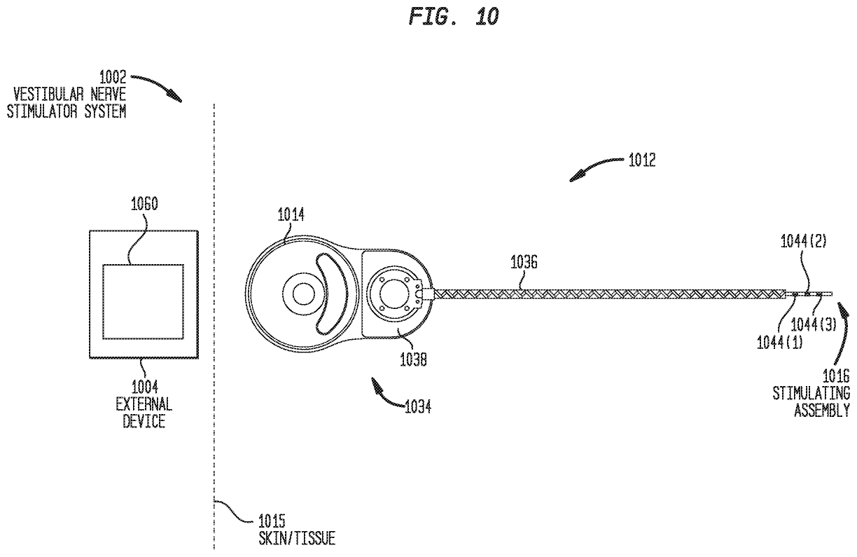

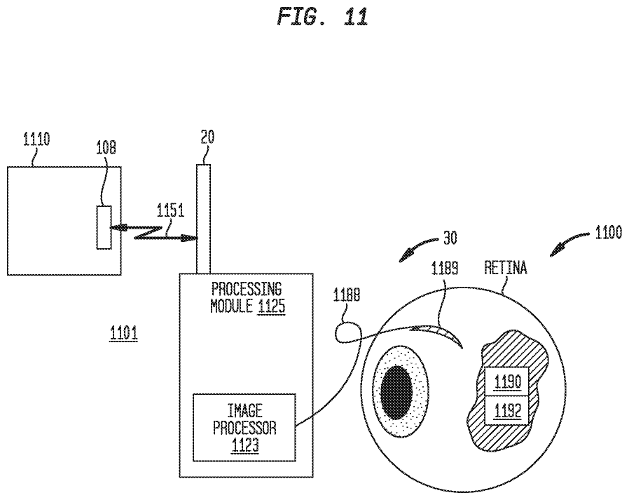

As previously described, the technology disclosed herein is not limited to cochlear implants, but instead can be applied in any of a variety of circumstances and with a variety of different medical devices and or other devices that provide electrical stimulation. Some further, non-limiting examples of medical devices that can benefit from technology disclosed herein are described in more detail below and are also depicted in FIGS. 9-11. As described below, the operating parameters for the devices described with reference to FIGS. 9-11 may be configured using a stimulating assembly with electrodes analogous that which has been previously described and depicted in FIGS. 1-8. For example, a stimulating assembly including electrodes as described herein can be used to prioritize clinician tasks associated with configuring the operating parameters of wearable medical devices, such as an implantable stimulation system as described in FIG. 9, a vestibular stimulator as described in FIG. 10, or a retinal prosthesis as described in FIG. 11. The devices and techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue.

FIG. 9 is a functional block diagram of an implantable stimulator system 900 that can benefit from the technologies described herein. The implantable stimulator system 900 includes the wearable device 900 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 902. Here, the wearable device 900 is configured to transcutaneously couple with the implantable device 30 via a wireless connection to provide additional functionality to the implantable device 30.

In the illustrated example, the wearable device 900 includes one or more sensors 912, a processor 914, a transceiver 918, and a power source 948. The one or more sensors 912 can be one or more units configured to produce data based on sensed activities. In an example where the stimulation system 900 is an auditory prosthesis system, the one or more sensors 912 include sound input sensors, such as a microphone, an electrical input for an FM hearing system, other components for receiving sound input, or combinations thereof. Where the stimulation system 900 is a visual prosthesis system, the one or more sensors 912 can include one or more cameras or other visual sensors. Where the stimulation system 900 is a cardiac stimulator, the one or more sensors 912 can include cardiac monitors. The processor 914 can be a component (e.g., a central processing unit) configured to control stimulation provided by the implantable device 30. The stimulation can be controlled based on data from the sensor 912, a stimulation schedule, or other data. Where the stimulation system 900 is an auditory prosthesis, the processor 914 can be configured to convert sound signals received from the sensor(s) 912 (e.g., acting as a sound input unit) into signals 951. The transceiver 918 is configured to send the signals 951 in the form of power signals, data signals, combinations thereof (e.g., by interleaving the signals), or other signals. The transceiver 918 can also be configured to receive power or data. Stimulation signals can be generated by the processor 914 and transmitted, using the transceiver 918, to the implantable device 30 for use in providing stimulation.

In the illustrated example, the implantable device 30 includes a transceiver 918, a power source 948, and a medical instrument 911 that includes an electronics module 910 and a stimulator assembly 930 (which includes an electrode assembly including electrodes based upon the technology as described herein). The implantable device 30 further includes a hermetically sealed, biocompatible implantable housing 902 enclosing one or more of the components.

The electronics module 910 can include one or more other components to provide medical device functionality. In many examples, the electronics module 910 includes one or more components for receiving a signal and converting the signal into the stimulation signal 915. The electronics module 910 can further include a stimulator unit. The electronics module 910 can generate or control delivery of the stimulation signals 915 to the stimulator assembly 930. In examples, the electronics module 910 includes one or more processors (e.g., central processing units or microcontrollers) coupled to memory components (e.g., flash memory) storing instructions that when executed cause performance of an operation. In examples, the electronics module 910 generates and monitors parameters associated with generating and delivering the stimulus (e.g., output voltage, output current, or line impedance). In examples, the electronics module 910 generates a telemetry signal (e.g., a data signal) that includes telemetry data. The electronics module 910 can send the telemetry signal to the wearable device 900 or store the telemetry signal in memory for later use or retrieval.

The stimulator assembly 930 can be a component configured to provide stimulation to target tissue. In the illustrated example, the stimulator assembly 930 is an electrode assembly that includes an array of electrode structures having features as described in one or more of the previous embodiments. The lead can be disposed proximate tissue to be stimulated. Where the system 900 is a cochlear implant system, the stimulator assembly 930 can be inserted into the recipient's cochlea. The stimulator assembly 930 can be configured to deliver stimulation signals 915 (e.g., electrical stimulation signals) generated by the electronics module 910 to the cochlea to cause the recipient to experience a hearing percept. In other examples, the stimulator assembly 930 can also include a vibratory actuator disposed inside or outside of a housing of the implantable device 30 and configured to generate vibrations. The vibratory actuator receives the stimulation signals 915 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 918 can be components configured to transcutaneously receive and/or transmit a signal 951 (e.g., a power signal and/or a data signal). The transceiver 918 can be a collection of one or more components that form part of a transcutaneous energy or data transfer system to transfer the signal 951 between the wearable device 900 and the implantable device 30. Various types of signal transfer, such as electromagnetic, capacitive, and inductive transfer, can be used to usably receive or transmit the signal 951. The transceiver 918 can include or be electrically connected to a coil 20.

As illustrated, the wearable device 900 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 948 can be one or more components configured to provide operational power to other components. The power source 948 can be or include one or more rechargeable batteries. Power for the batteries can be received from a source and stored in the battery. The power can then be distributed to the other components as needed for operation.

As should be appreciated, while particular components are described in conjunction with FIG.9, technology disclosed herein can be applied in any of a variety of circumstances. The above discussion is not meant to suggest that the disclosed techniques are only suitable for implementation within systems akin to that illustrated in and described with respect to FIG. 9. In general, additional configurations can be used to practice the methods and systems herein and/or some aspects described can be excluded without departing from the methods and systems disclosed herein.

FIG. 10 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012,

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 135 also includes an internal/implantable coil 1014 that is generally external to the housing 1038, but which is connected to the transceiver via a hermetic feedthrough (not shown).

The stimulating assembly 1016 comprises a plurality of electrodes 1044 (1)-(3) disposed in a carrier member (e.g., a flexible silicone body). The carrier member and electrodes can have the same or similar configurations as previously described herein. In this specific example, the stimulating assembly 1016 comprises three (3) stimulation electrode structures, referred to as electrode structures 1044(1), 1044(2), and 1044(3). The electrode structures 1044(1), 1044(2), and 1044(3) function as an electrical interface for delivery of electrical stimulation signals to the recipient's vestibular system and can have an arrangement as described above.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 1012, the external device 1004, and/or another external device, can be configured to implement the techniques presented herein. That is, the vestibular stimulator 1012, possibly in combination with the external device 1004 and/or another external device, can include an evoked biological response analysis system, as described elsewhere herein.

FIG. 11 illustrates a retinal prosthesis system 1101 that comprises an external device 1110 (which can correspond to the wearable device 900) configured to communicate with a retinal prosthesis 1100 via signals 1151. The retinal prosthesis 1100 comprises an implanted processing module 1125 (e.g., which can correspond to the implantable device 30) and a retinal prosthesis sensor-stimulator 1190 is positioned proximate the retina of a recipient. The external device 1110 and the processing module 1125 can communicate via coils 108, 20.

In an example, sensory inputs (e.g., photons entering the eye) are absorbed by a microelectronic array of the sensor-stimulator 1190 that is hybridized to a glass piece 1192 including, for example, an embedded array of microwires. The sensor-stimulator 1190 can include an array of electrode structures, as described above, coupled with a carrier member having configurations as previously described herein. The glass can have a curved surface that conforms to the inner radius of the retina. The sensor-stimulator 1190 can include a microelectronic imaging device that can be made of thin silicon containing integrated circuitry that convert the incident photons to an electronic charge.

The processing module 1125 includes an image processor 1123 that is in signal communication with the sensor-stimulator 1190 via, for example, a lead 1188 which extends through surgical incision 1189 formed in the eye wall. In other examples, processing module 1125 is in wireless communication with the sensor-stimulator 1190. The image processor 1123 processes the input into the sensor-stimulator 1190 and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire and a signal is sent to the optic nerve, thus inducing a sight perception.

The processing module 1125 can be implanted in the recipient and function by communicating with the external device 1110, such as a behind-the-ear unit, a pair of eyeglasses, etc. The external device 1110 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

In a further embodiment, a stimulating assembly with electrode structures as described herein can be applied for use in devices that provide electroporation of cells for the purpose of DNA transfection. The electrode structure configuration as described herein, where the insulating skirt surrounds the conductive pad so as to limit a direction of current flow to a precisely focused or targeted area along a surface that is coupled with the conductive pad, facilitates the application of a sufficiently high electrical pulses to body tissues in order to achieve injection of nucleic acids and/or other chemical compounds through electrically induced pores in cell membranes and into cells of the body tissues.

Further, technology described herein can also be applied to consumer devices. These different systems and devices can benefit from the technology described herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. An apparatus, comprising:
a carrier member comprising an electrically insulating material;
at least one electrode contact;
at least one electrically conductive polymer pad disposed on the at least one electrode contact; and
at least one electrically insulating polymer skirt at least partially surrounding the at least one electrically conductive polymer pad.

2. The apparatus of claim 1, wherein the at least one electrically conductive polymer pad and the at least one electrically insulating polymer skirt include complimentary surfaces that define an engaging interface between the at least one electrically conductive polymer pad and the at least one electrically insulating polymer skirt.

3. The apparatus of claim 1, wherein the at least one electrically conductive polymer pad has a cross-sectional dimension that varies over a lengthwise dimension of the at least one electrically conductive polymer pad.

4. The apparatus of claim 1, wherein the at least one electrically insulating polymer skirt has a cross-sectional dimension that varies over a lengthwise dimension of the at least one electrically insulating polymer skirt.

5. The apparatus of claim 1, wherein the at least one electrically conductive polymer pad has a frustoconical shape.

6. The apparatus of claim 1, wherein the at least one electrically insulating polymer skirt comprises an annular member, and the at least one electrically conductive polymer pad is at least partially disposed within a space defined by the annular member.

7. The apparatus of claim 6, wherein the at least one electrically insulating polymer skirt comprises a compressive polymer material with a free end that compresses and conforms to a surface to which the free end engages while surrounding the at least one electrically conductive polymer pad so as to limit electrical current flow from the at least one electrically conductive polymer pad to an area of the surface bounded by the free end of the at least one electrically insulating polymer skirt.

8. The apparatus of any of claim 1, wherein the at least one electrically conductive polymer pad and the at least one electrically insulating polymer skirt include complimentary engaging surfaces that are curved in shape so as to define a curved interface between the complimentary engaging surfaces.

9. The apparatus of claim 8, wherein a surface portion of the at least one electrically conductive polymer pad that defines part of the complimentary engaging surfaces has a concave shape.

10. The apparatus of any of claim 1, wherein the at least one electrically conductive polymer pad includes a free end that is not covered by and is coplanar with a free end portion of the at least one electrically insulating polymer skirt.

11. The apparatus of claim 1, wherein the at least one electrode contact comprises a metal material, and wherein the at least one electrode contact is electrically coupled with the at least one electrically conductive polymer pad.

12. The apparatus of claim 11, wherein the at least one electrode contact is at least partially disposed within the carrier member.

13. (canceled)

14. (canceled)

15. (canceled)

16. (canceled)

17. The apparatus of claim 11, wherein the at least one electrode contact further comprises a mesh material that extends from the carrier member into the at least one electrically conductive polymer pad.

18. The apparatus of claim 1, wherein the at least one electrically conductive polymer pad comprises a hydrogel material.

19. The apparatus of claim 18, further comprising a therapeutic substance disposed within the at least one electrically conductive polymer pad.

20. The apparatus of claim 19, further comprising a dissolvable barrier coupled with the at least one electrically conductive polymer pad, wherein dissolution of the dissolvable barrier facilitates elution of the therapeutic substance from the at least one electrically conductive polymer pad.

21. The apparatus of claim 1, wherein the at least one electrode contact comprises a plurality of electrode contacts, and wherein each of the plurality of electrode contacts is associated with a corresponding one of a plurality of electrically conductive polymer pads and a corresponding one of a plurality of electrically insulating polymer skirts.

22. The apparatus of claim 21, wherein the carrier member comprises an elongated structure, and the plurality of electrode contacts are disposed in a consecutively aligned arrangement along the elongated structure.

23. The apparatus of claim 21, wherein the plurality of electrode contacts are aligned in rows and columns along the carrier member.

24. A system comprising the apparatus of claim 1, and an electrical stimulator that provides electrical current to the apparatus.

25. The system of claim 24, wherein the system comprises a cochlear implant device, the apparatus comprises a perimodiolar stimulating assembly, and the carrier member is configured to wrap around a modiolus when implanted within a cochlea of a recipient.

26. A method comprising:
inserting a stimulating assembly into a body chamber of a recipient, the stimulating assembly comprising a carrier member and an electrode structure coupled with the carrier member, the carrier member comprising an electrically insulating material, and the electrode structure comprising an electrically conductive polymer pad and an electrically insulating polymer skirt at least partially surrounding the electrically conductive polymer pad; and
facilitating current flow from the electrode structure to nerve cells within the body chamber.

27. The method of claim 26, wherein the electrically insulating polymer skirt comprises a compressive polymer material and has an annular shape within which the electrically conductive polymer pad is disposed, and wherein inserting further comprises:
facilitating compression of the electrically insulating polymer skirt such that a free end of the electrically insulating polymer skirt engages and conforms with a surface of the body chamber while surrounding the electrically conductive polymer pad.

28. The method of claim 27, wherein facilitating current flow further comprises:
substantially limiting current flow from a free end of the electrically conductive polymer pad to only a surface of the body chamber that is bounded by the free end of the electrically insulating polymer skirt.

29. The method of claim 26, wherein the stimulating assembly comprises a perimodiolar stimulating assembly, and wherein inserting comprises:
inserting the stimulating assembly into a cochlea of the recipient such that the carrier member wraps around a modiolus within the cochlea.

30. (canceled)

31. (canceled)

32. (canceled)
